(19) United States
(12) Patent Application Publication
(10) Pub. No.: US 2004/0142095 A1
(43) Pub. Date: Jul. 22, 2004

(54) Nucleic acid arrays and method for detecting nucleic acids by using nucleic acid arrays

(75) Inventors: Masatoshi Narahara, Sayama (JP); Toshiro Saito, Hatoyama (JP); Hiroyuki Tomita, Tachikawa (JP); Hirokazu Kato, Hatoyama (JP)

Correspondence Address: REED SMITH LLP, Suite 1400, 3110 Fairview Park Drive, Falls Chuch, VA, 22042, US

(73) Assignee: Hitachi, Ltd.

(21) Appl. No.: 10/761,208

(22) Filed: Jan. 22, 2004

Related U.S. Application Data
(63) Continuation of application No. 09/942,563, filed on Aug. 31, 2001.

(30) Foreign Application Priority Data
Jan. 9, 2001 (JP) 2001 - 001761

(51) Int. Cl. A61L 2/00; B05D 3/00; C12Q 1/68

(52) U.S. Cl. 427/2.11

(57) ABSTRACT

The present invention provides nucleic acid arrays with improved sensitivity for a nucleic acid. The array comprises various kinds of nucleic acid probes, which are capable of hybridizing to the nucleic acid, immobilized at different positions on a substrate. Single-stranded nucleic acid probes are immobilized on the substrate by covalent bond, and functional groups that can have negative charge by dissociating in an aqueous solution or by hydrolysis are introduced on the surface of regions of the substrate on which no nucleic acid probe is immobilized.

### FIELD OF THE INVENTION

The present invention relates to nucleic acid arrays for detecting nucleic acids by hybridization and a method for detecting the nucleic acids using the arrays. Particularly, the present invention provides nucleic acid arrays in which sensitivity for the nucleic acids is enhanced by increasing the amount of hybridization of nucleic acids and decreasing noise by suppressing adsorption of nucleic acids to regions on which no nucleic acid probe is immobilized; and a method for detecting nucleic acids using the arrays.

### BACKGROUND OF THE INVENTION

In recent years, microarray technology has become of major interest to profile gene expression. Arrays enable simultaneous observation of expression of several thousands or several tens of thousands of genes. The principle of arrays is to immobilize several types of nucleic acid probes on a substrate and to allow labeled nucleic acids to hybridize thereto. Nucleic acids having a complementary base sequence to nucleic acid probes hybridize specifically to arrayed probe molecules. Then, measurement of signals of the labeled nucleic acid hybridizing to the nucleic acid probes enables identification of the nucleic acid hybridizing and measurement of amount of hybridizing molecules. When fluorescently-labeled nucleic acids are used, the amount of hybridizing molecules is obtained by subtracting background, which is the fluorescent signal value for a region without hybridization, from the fluorescent signal value for a region with hybridization. Therefore in this case, sensitivity can be improved by increasing the fluorescent signal value for a region with hybridization and lowering the background value. A method for producing such arrays disclosed in U.S. Pat. No. 5807522 involves spotting double-stranded cDNA probes with a spotter very densely on a substrate coated with resin having an amino group, thermally denaturating the double-stranded cDNA probes, and treating regions on which no cDNA probe is immobilized with succinic anhydride, thereby blocking adsorption of nucleic acids upon hybridization in the regions.

However, arrays of U.S. Pat. No. 5807522 require the use of a probe with long chain length because double-stranded cDNA probes are immobilized by weak electrostatic bond between amino groups on the substrate and the probes. Another problem of the arrays is decreased sensitivity for nucleic acids because cDNA probes may be stripped off upon blocking treatment or hybridization. Further, thermal denaturation is performed after immobilization of cDNA probes so that not only sense strands derived from nucleic acid probes but also antisense strands remain on the substrate. Since antisense strands are kept immobilized near their corresponding sense strands, hybridization of the sense and the antisense strands proceeds competitively with hybridization of the sense strands and nucleic acids thereby significantly lowering hybridization efficiency.

A method which enables highly efficient hybridization and causes no stripping of nucleic acid probes has been reported in “Nucleic Acids Research (Vol. 24, pp.3031, 1996).” This method involves immobilizing previously-synthesized single-stranded nucleic acid probes on a substrate by covalent bond. However, nucleic acid targets may adsorb to regions on which no nucleic acid probe is immobilized, resulting in a high background. A method of Japanese Patent Laid open Publication No. 11-187900 involves immobilizing single-stranded probes by covalent bond, and allowing Bovine Serum Albumin to adsorb to regions on which no nucleic acid probe is immobilized, so as to block adsorption of nucleic acids. However, the large molecular weight of Bovine Serum Albumin will be a steric hindrance when nucleic acids approach nucleic acid probes during hybridization, thereby lowering hybridization efficiency.

As described above, it has been difficult to stably bind nucleic acid probes on a substrate, improve hybridization efficiency, and increase sensitivity. The present invention provides nucleic acid arrays and a method for detecting nucleic acids by using nucleic acid arrays, in which stripping of nucleic acid probes can be prevented and hybridization efficiency can be improved by immobilized single-stranded nucleic acid probes by covalent bond, and in which adsorption of nucleic acid targets in the surface of regions on which no nucleic acid probe is immobilized can be prevented to increase sensitivity for the targets by introducing functional groups that can have negative charge by dissociating in an aqueous solution or functional groups negatively charged by hydrolysis are introduced onto the surface.

### SUMMARY OF THE INVENTION

To achieve the above purposes, nucleic acid arrays of the present invention comprise various kinds of single-stranded nucleic acid probes which are capable of hybridizing to nucleic acids and which are immobilized at different positions on a substrate, wherein: the single-stranded nucleic acid probes are immobilized by covalent bond on the substrate; and functional groups which can have negative charge by dissociating in an aqueous solution are present on the surface of regions of the substrate on which no nucleic acid probe is immobilized. The use of single-stranded nucleic acid probes improves hybridization efficiency, while immobilization by covalent bond of single-stranded nucleic acid probes could prevent stripping of nucleic acid probes during hybridization. Moreover, introduction of functional groups that can have negative charge by dissociating in an aqueous solution onto the surface of regions of the substrate on which no nucleic acid probe is immobilized can prevent adsorption of nucleic acids using electrostatic repulsion between negatively charged nucleic acids and the introduced functional groups.

Furthermore, the nucleic acid arrays of the present invention are characterized by introducing functional groups that can have negative charge by dissociating in an aqueous solution onto regions of the substrate on which no nucleic acid probe is immobilized. This can be achieved by immobilizing single-stranded nucleic acid probes on a substrate by covalent bond, and then immobilizing by covalent bond a compound with a functional group which can have negative charge by dissociation onto regions of the substrate on which no single-stranded nucleic acid probe is immobilized. Such functional groups introduced by covalent bond are not easily stripped off during hybridization, so that adsorption of nucleic acids can be more efficiently prevented.

Moreover, the nucleic acid arrays of the present invention are characterized by introducing functional groups that can have negative charge by dissociating in an aqueous solution onto regions of the substrate on which no nucleic acid probe is immobilized. This can be achieved by immobilizing single-stranded nucleic acid probes on the substrate by covalent bond, and then immobilizing by hydrophobic bond a compound with a functional group which can have negative charge by dissociation onto regions of the substrate on which no single-stranded nucleic acid probe is immobilized. The use of hydrophobic bond enables introduction of functional groups that can have negative charge by dissociation regardless of the type of functional group on the substrate.

The nucleic acid arrays of the present invention wherein various single-stranded nucleic acid probes which are capable of hybridizing to nucleic acids are immobilized at different positions on a substrate, are characterized in that the single-stranded nucleic acid probes are immobilized on a substrate by covalent bond; and functional groups that are negatively charged by hydrolysis are present on the surface of regions of the substrate on which no nucleic acid probe is immobilized. First, functional groups that can react to functional groups of nucleic acid probes on the substrate surface are introduced, and then the introduced functional groups and the functional groups of nucleic acid probes are allowed to react to each other, thereby immobilizing the nucleic acid probes. Following immobilization of the nucleic acid probes, functional groups that can have negative charge in an aqueous solution are generated by hydrolysing unreacted functional groups. Since this method enables introduction of a functional group that can have negative charge without using additional compound, the production cost of nucleic acid arrays can be reduced.

Further, the method of the present invention for detecting nucleic acids is characterized by using nucleic acid arrays in which various single-stranded nucleic acid probes are immobilized by covalent bond at different positions on a substrate; and functional groups that can have negative charge by dissociation in an aqueous solution or those negatively charged by hydrolysis are present on the surface of regions of the substrate on which no nucleic acid probe is immobilized. The nucleic acid arrays with high sensitivity enable detection of target nucleic acid with high reproducibility and reliability.

### BRIEF DESCRIPTION OF THE DRAWINGS

FIG. 1 is a diagrammatic illustration of an example of a method for producing nucleic acid arrays of the present invention and their structure.

FIG. 2 is a graph showing the intensity of fluorescence after hybridization of a region where nucleic acid probes obtained in examples 1-8 and comparative examples 1-3 are immobilized.

FIG. 3 is a graph showing the intensity of fluorescence after hybridization of a region where nucleic acid probes obtained in examples 1-8 and comparative examples 1-3 are not immobilized.

FIG. 4 is a schematic illustration showing one example of nucleic acid arrays of the present invention.

FIG. 5 is a Scatter plot of the intensity of fluorescence of 200 spots obtained in Example 9 in which the intensity of fluorescence of the first experiment is located on the horizontal axis and that of the second experiment is located on the vertical axis.

FIG. 6 is a Scatter plot of the intensity of fluorescence of 200 spots obtained in Example 10 in which the intensity of fluorescence of the first experiment is located on the horizontal axis and that of the second experiment is located on the vertical axis.

FIG. 7 is a Scatter plot of the intensity of fluorescence of 200 spots obtained in Example 11 in which the intensity of fluorescence of the first experiment is located on the horizontal axis and that of the second experiment is located on the vertical axis.

FIG. 8 is a Scatter plot of the intensity of fluorescence of 200 spots obtained in Comparative example 1 in which the intensity of fluorescence of the first experiment is located on the horizontal axis and that of the second experiment is located on the vertical axis.

### Definitions for Number Signs

1 slide glass

2 spot

### DETAILED DESCRIPTION OF THE INVENTION

Detailed description of the present invention will be given as follows.

In the present invention, single-stranded nucleic acid probes are immobilized on a substrate by covalent bond, and then functional groups that can have negative charge by dissociating in an aqueous solution or those negatively charged by hydrolysis are introduced onto regions of the substrate on which no nucleic acid probe is immobilized. The aqueous solution which allows dissociation or hydrolysis of functional groups is not specifically limited in this specification, but preferably is in a pH range of 6.0 to 8.0.

Examples of single-stranded nucleic acid probes and nucleic acids used in the present invention are not specifically limited so far as they can hybridize to each other. The term “hybridization” means that two nucleic acids having complementary sequences form a double-stranded hybrid by hydrogen bond. Such combinations of two nucleic acids include DNA/DNA, DNA/RNA, RNA/RNA, DNA/PNA, RNA/PNA and PNA/PNA.

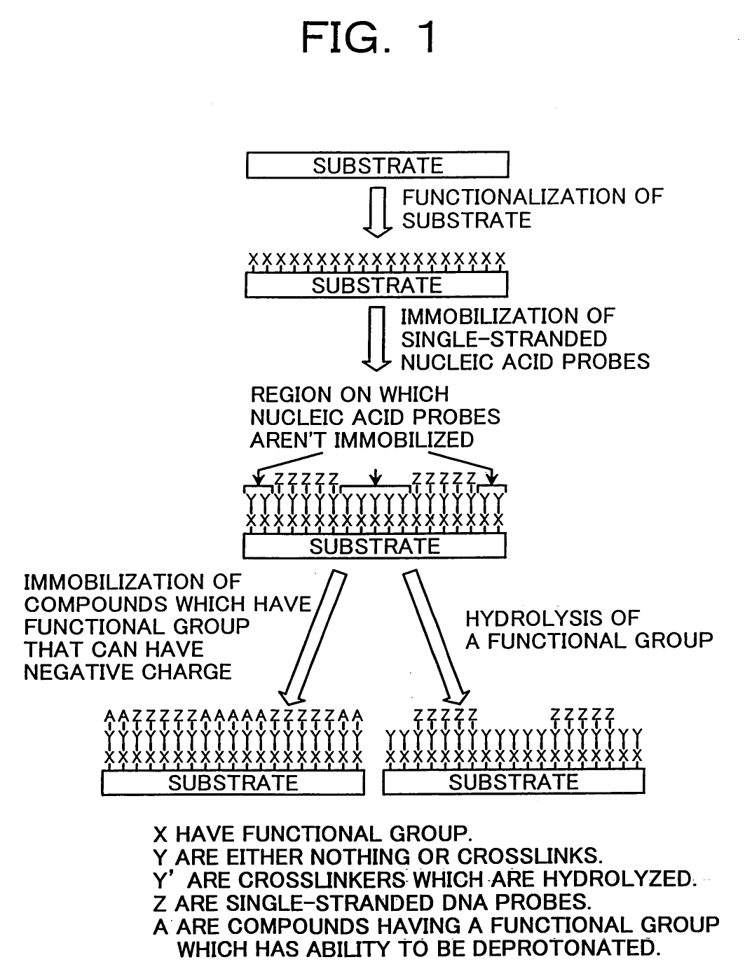

An example of a method for immobilizing single-stranded nucleic acid probes on a substrate which can be used in the present invention is a method comprising introducing functional groups which can react to both nucleic acid probes and a substrate, and binding them (see FIG. 1). Examples of a functional group that can be introduced into a nucleic acid probe terminus include an amino group and a thiol group. In addition, a method for introducing functional groups that can react to nucleic acid probes onto a substrate is, for example a method using various crosslinkers. A crosslinker reacts with a first functional group of a substrate (denoted as X in FIG. 1), and then a second functional group that can react with a functional group of the nucleic acid probes (denoted as Y in FIG. 1) is introduced. When nucleic acid probes having amino groups introduced therein are used, examples of the second functional group include an isothiocyanate group, an isocyanate group, an imidoester group, and an N-hydroxysuccinimide group. When nucleic acid probes having thiol groups introduced therein are used, examples include a haloacetyl group, a maleimide group and a disulfide group. When both the first functional group and that of nucleic acid probes are amino groups, examples of a crosslinker used herein include bifunctional N-hydroxysuccinimides such as DSG (Disuccinimidyl glutarate); diisocyanates, such as 1,4-phenylenediisocyanate; and diisothiocyanates, such as 1,4-phenylenediisothiocyanate; or a bifunctional crosslinker containing two different functional groups of the above. When the first functional group is an amino group and that of nucleic acid probes is a thiol group, examples of crosslinkers include a bifunctional crosslinker having functional groups which can react with an amino group or a thoil group, for example a bifunctional compound having an N-hydroxysuccinimide group and a maleimido group, such as GMBS (N-(γ-Maleimidobutyryloxy)succinimide ester); a bifunctional compound having an N-hydroxysuccinimide group and a haloacetyl group, such as SIAB (N-Succinimidyl (4-iodoacetyl) aminobenzoate); a bifunctional compound having an N-hydroxysuccinimide group and a disulfide group, such as SPDP (N-Succinimidyl-3-(2-pyridyldithio)-propionate).

Examples of materials with suitable qualities of a substrate used in this invention include one or more materials selected from plastics, inorganic polymers, metal, natural polymer and ceramic. Examples of plastics include polyethylene, polystyrene, polycarbonate, polypropylene, polyamide, phenol resin, epoxy resin, polycarbodiimide resin, polyvinyl chloride, polyvinylidene fluoride, polyethylene fluoride, polyimide and acrylate resin. Examples of inorganic polymers include glass, crystal, carbon, silica gel, and graphite. Examples of metal include those metals that are solid under room temperature, such as gold, platinum, silver, copper, iron, aluminum, and magnet. Examples of ceramic include alumina, silica, silicon carbide, silicon nitride, and boron carbide. The shape of the above substrate is not specifically limited. When nucleic acids are detected with fluorescence, a substrate is preferably the shape of a smooth plate in order to prevent scattering of excitation light.

Examples of methods for introducing a first functional group which can react with the crosslinker used in the present invention onto a substrate include a method which comprises coating a resin having a functional group over a substrate and a method which comprises chemically treating the surface of a substrate. Resin to be coated is not specifically limited. For example,preferred resin has an amino group which may form a stable bond with a crosslinker, such as poly-L-lysine. Alternatively, after coating with resin containing no amino group, such as polyimide or polystyrene, plasma treatment may be performed in an atmosphere of nitrogen so as to introduce an amino group. Examples of a method for introducing a first functional group using chemical treatment include a method which comprises applying a silane coupling agent over silicon compounds such as glass and silicon nitride, or metal oxides, and a method which comprises treating a substrate having a gold film on its top surface using alkane thiols.

In the present invention, first functional groups introduced on a substrate without a crosslinker and those of nucleic acid probes may be allowed to directly react to each other so as to immobilize the nucleic acid probes. For example, a compound having an aldehyde group, such as glutaraldehyde is coated on a substrate, and then nucleic acid probes with amino groups are immobilized. Alternatively, a substrate is chemically treated with a silane coupling agent having an epoxy group so that nucleic acid probes having amino groups can be immobilized.

Furthermore in the present invention, first, nucleic acid probes are immobilized on a substrate, and then functional groups (denoted as “A” in FIG. 1) that can have negative charge in an aqueous solution are introduced onto regions of the substrate on which no nucleic acid probe is immobilized. Methods for introducing functional groups that can have negative charge include three methods as follows. The first method involves immobilizing compounds having functional groups that can have negative charge onto regions on which no nucleic acid probe is immobilized by covalent bond (Flow on the left in FIG. 1). The second method involves immobilizing amphiphilic molecules such as a surfactant onto regions on which no nucleic acid probe is immobilized by hydrophobic bond (Flow on the left in FIG. 1). The third method involves introducing functional groups that can have negative charge by hydrolyzing the functional groups on a substrate (Flow on the right in FIG. 1).

Compounds to be immobilized by covalent bond in the first method (A in FIG. 1) contain both functional groups for reacting with functional groups on a substrate and functional groups that can have negative charge. Examples of functional groups for reacting with functional groups on a substrate are not specifically limited so far as they can react with those on a substrate and form covalent bonds. More specifically, preferred examples include an amino group and a thiol group, which can react with functional groups introduced on a substrate using a crosslinker so as to form more stable covalent bonds. On the other hand, examples of functional groups that can have negative charge are not specifically limited so far as they can have negative charge by dissociating in an aqueous solution. A preferred example is a carboxyl group with a large dissociation coefficient. Examples of single molecules having both of these two functional groups include various amino acids, such as alanin and glycine having an amino group and a carboxyl group, and cysteine having a thiol group and a carboxyl group.

The second method using hydrophobic bond involves immersing a substrate on which nucleic acid probes (Z in FIG. 1) have been immobilized in an aqueous solution containing amphiphilic molecules (A in FIG. 1) having hydrophilic groups and hydrophobic groups in their molecules. At this time, functional groups that can have negative charge are introduced on regions on which no nucleic acid probe is immobilized by hydrophobic bonds between the functional groups on the substrate and the hydrophobic groups of the amphiphilic molecules. Examples of amphiphilic molecules used in the present invention are not specifically limited so far as they have anionic dissociation groups that can have negative charge by dissociating in an aqueous solution. Such anionic dissociation groups include carboxyl groups, sulfonic acid groups, hydrogen sulfide groups and salts thereof. Examples of hydrophobic groups are not specifically limited, including long alkyl chains, aromatic rings, or hydrophobic groups containing one or more of the above.

The third method using hydrolysis involves introducing functional groups (Y in FIG. 1) which can react with those of nucleic acid probes onto a substrate, followed by immobilizing the nucleic acid probes (Z in FIG. 1) by covalent bond. Subsequently, functional groups that can have negative charge in an aqueous solution (Y′ in FIG. 1) are introduced by immersing the substrate in an aqueous solution with an appropriate pH so as to hydrolyze the functional groups on regions on which no nucleic acid probe is immobilized. Examples of such functional groups are not specifically limited so far as they can react with functional groups of nucleic acid probes and can be converted to those which can have negative charge by hydrolysis. Such functional groups include a N-hydroxysuccinimide group and a maleimide group.

Examples of the method for detecting nucleic acids used in the present invention are not specifically limited so far as it can detect labeled nucleic acids. Examples of such a detection method are methods using fluorescence, phosphorescence, emission or radioisotopes. A method for detecting unlabeled nucleic acids that may be used herein involves interchelating special compounds to double-stranded nucleic acids formed by hybridization, and detecting the compounds with their emission or detecting them electrically, thereby detecting hybridization amount.

Now the present invention will be further explained-with examples as follows.

### EXAMPLE

### Example 1

(1) Washing of a Substrate

A commercially available slide glass (Gold Seal Brand) was immersed in an alkaline solution (sodium hydroxide; 50 g, distilled water; 150 ml, 95% ethanol; 200 ml) at room temperature for 2 hours. Then the slide glass was transferred into distilled water, rinsed three times, there by completely removing alkaline solution.

(2) Introduction of Functional Groups for Immobilizing Nucleic Acid Probes

The washed slide glass was immersed in 10% poly-L-lysine (Sigma) solution for 1 hour, and then the slide glass was taken out and subjected to centrifugation at 500 r.p.m. for 1 min using a centrifugal separator for microtiter plates, to remove poly-L-lysine solution. Then, the slide glass was set in a vacuum incubator, dried at 40° C. for 5 min, thereby introducing amino groups on the slide glass. Subsequently, the slide glass having amino groups introduced thereon was immersed in 1 mM GMBS (PIERCE) dimethyl sulfoxide solution for 2 hours, washed with dimethyl sulfoxide, thereby introducing maleimide groups on the slide glass surface.

(3) Immobilization of Single-stranded Nucleic Acid Probes

Nucleic acid probes 1 having thiol groups introduced therein were synthesized using a DNA synthesizer (Applied Biosystem, model 394). Then, the nucleic acid probes were purified by high performance liquid chromatography. Next, 1 μl of the synthesized and purified 2 μM nucleic acid probes, 4 μl of HEPES buffer solution (N-2-hydroxyethyl piperazine-N′-2-ethane sulfonic acid; 10 mM, pH6.5) and 5 μl of an addition agent (ethylene glycol) were mixed to prepare a spotting solution. The prepared spotting solution was spotted with a spotter (Hitachi software, SPBIO 2000) on arbitrary points on the slide glass, and allowed to stand for 2 hours at room temperature, thereby immobilizing the nucleic acid probes on the slide glass.

Nucleic Acid Probe 1;

HS—(CH₂)₆—O—PO₂—O—5′-GACACAGCAGGTCAAGAGGAGTACA-3′ (SEQ ID NO: 1)

(4) Introduction of Functional Groups that can have Negative Charge

The slide glass on which nucleic acid probes had been immobilized was immersed in 100 mM cysteine (Wako Pure Chemical Industries, Ltd) solution that had been adjusted to have pH 6.5 with a HEPES buffer solution for 2 hours . Thus, functional groups that can have negative charge by dissociation were introduced using covalent bond onto regions on which no nucleic acid probe had been immobilized.

(5) Hybridization Reaction

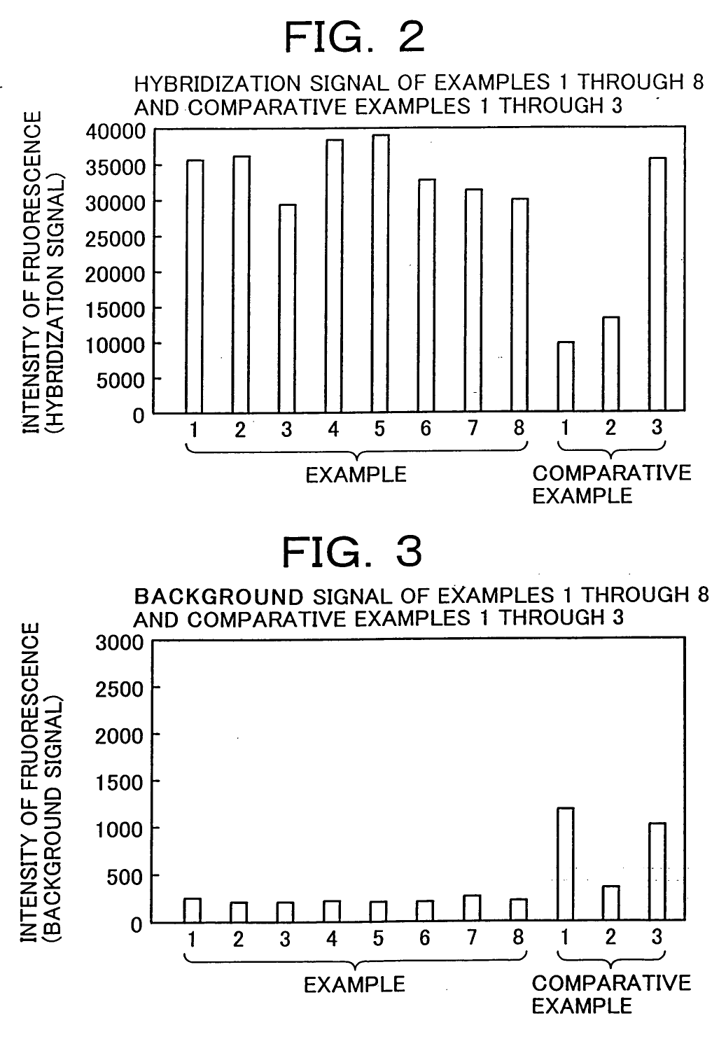

A nucleic acid having a complementary base sequence to the nucleic acid probe 1 and having 5′-end fluorescent-labeled with Texas red was synthesized using a DNA synthesizer. Next, a hybridization solution was prepared by addition of 8 μl of the 0.1 μM nucleic acid, 1.7 μl of 20×SSC (Wako Pure Chemical Industries, Ltd), and 0.3 μl of 10% sodium dodecyl sulfate solution(Lifetec Oriental). Then, the prepared hybridization solution was dropped onto the slide glass, covered with a cover glass, and then allowed to stand in a thermostatically controlled chamber at 40° C. for 12 hours for hybridization reaction to proceed. After hybridization reaction, the slide glass was immersed (and the cover glass was removed) in a mixture of 10× diluent of 20×SSC and 300× diluent of 10% sodium dodecyl sulfate solution, followed by washing with 100× diluent of 20×SSC. After that water was removed from the slide glass using a centrifugal separator for microtiter plates, fluorescence intensity of regions on which the nucleic acid probes had been immobilized (hybridization signal) and fluorescence intensity of regions on which no nucleic acid probe had been immobilized (background signal) were measured using a scanner for arrays (GSI Lumonics, Scan Array 5000). FIGS. 2 and 3 show the results.

In this example, after immobilization of single-stranded nucleic acid probes by covalent bond, carboxyl groups that can have negative charge by dissociating in an aqueous solution were introduced by covalent bond onto the surface of regions on which no nucleic acid probe had been immobilized. Nucleic acid probes were not stripped off during hybridization reaction and adsorption of nucleic acids could be suppressed. Therefore, high hybridization signal was obtained and lower background signal was achieved.

### Example 2

Example 2 was conducted by the same steps as in Example 1 except that step (4) “introduction of functional groups that can have negative charge” was changed as shown below.

(4) Introduction of Functional Groups that can have Negative Charge

The slide glass on which nucleic acid probes had been immobilized was immersed in 10 mM sodium dodecyl sulfate (Wako Pure Chemical Industries, Ltd) for 2 hours.

In this example, after immobilization of single-stranded nucleic acid probes by covalent bond, hydrogensulfate groups that can have negative charge by dissociating in an aqueous solution were introduced by hydrophobic bond onto the surface of regions on which no nucleic acid probe had been immobilized. Similar to Example 1, both high hybridization signal and suppressed background signal were achieved.

### Example 3

Example 3 was conducted by the same steps as in Example 1 except that step (4) “introduction of functional groups that can have negative charge” was changed as shown below.

(4) Introduction of Functional Groups that can have Negative Charge

The slide glass on which nucleic acid probes had been immobilized was immersed in an EPPS buffer solution (3-[4-(2-hydroxyethyl)-1-piperazinyl]propane sulfonate; 50 mM, pH8.0).

In this example, after immobilization of single-stranded nucleic acid probes by covalent bond, the nucleic acid probe-immobilized substrate was immersed in an alkaline solution so as to hydrolyze maleimide groups, and functional groups that can have negative charge in an aqueous solution were introduced onto the surface of regions on which no nucleic acid probe had been immobilized. Similar to Example 1, both high hybridization signal and suppressed background signal were achieved.

### Example 4

Example 4 was conducted by the same steps as in Example 1 except that step (2) “introduction of functional groups to immobilize nucleic acid probes” was changed as shown below. (2) Introduction of Functional Groups for Immobilizing Nucleic Acid Probes

The washed slide glass was immersed in 1% 3-aminopropyl triethoxy silane (Aldrich) solution in 95% ethanol for 1 hour. Then, the slide glass was taken out, and then centrifuged at 500 r.p.m. for 1 min using a centrifugal separator for microtiter plates to remove the reaction solution. Next, the slide glass was set in a vacuum thermostat and baked at 120° C. for 1 hour, thereby introducing amino groups onto the slide glasses. Further, the amino group-introduced slide glass was immersed in 1 mM GMBS dimethyl sulfoxide solution for 2 hours, and then washed with dimethyl sulfoxide.

In this example, functional groups that can react with a crosslinker were introduced on a substrate by different methods from those in Examples 1 to 3, and then single-stranded nucleic acid probes were immobilized via a crosslinker in the same manner as in Examples 1 to 3. Subsequently, as in Example 1, carboxyl groups that can have negative charge by dissociating in an aqueous solution were introduced by covalent bond onto the surface of regions on which no nucleic acid probe had been immobilized. In this example, both stripping of nucleic acid probes and adsorption of nucleic acids could be prevented similar to Example 1, so that higher hybridization signal and lower background signal were achieved.

### Example 5

Example 5 was conducted by the same steps as in Example 4 except that step (4) “introduction of functional groups that can have negative charge” was conducted in the same manner as in Example 2.

In this example, after functional groups were introduced onto a substrate by the method of Example 4 and single-stranded nucleic acid probes were immobilized, hydrogensulfate groups that can have negative charge by dissociating in an aqueous solution were introduced by hydrophobic bond onto the surface of regions on which no nucleic acid probe had been immobilized according to the method of Example 2. Similar to Example 1, both high hybridization signal and low background signal were achieved.

### Example 6

Example 6 was conducted by the same steps as in Example 4, except that step (4) “introduction of a functional group that can have negative charge” was performed in the same manner as in Example 3.

In this example, after functional groups were introduced on a substrate by the method described in Example 4, and single-stranded nucleic acid probes were immobilized thereto, the substrate on which the nucleic acid probes were immobilized was immersed in an alkaline solution to hydrolyze a maleimide group, thereby introducing a functional group that can have negative charge in an aqueous solution to the surface of a region where no nucleic acid probe was immobilized. Similar to Example 1, both high hybridization signal and low background signal were achieved.

### Example 7

Example 7 was conducted by the same steps as in Example 1 except that step (2) introduction of functional groups for immobilizing nucleic acid probes, (3) immobilization of single-stranded nucleic acid probes and (4) introduction of functional groups that can have negative charge were altered as follows.

(2) Introduction of Functional Groups for Immobilizing Nucleic Acid Probes

The washed slide glass was immersed for 1 hour in 95% ethanol solution of 1% 3-glycidoxypropyltrimethoxysilane (manufactured by Aldrich), and then the slide glass was taken out and subjected to centrifugation for one minute at 500 r.p.m. using a centrifugal separator for microtiter plates, thereby removing the reaction solution. Next, the slide glass was put in a suction thermostat and baked for an hour at 120° C. to introduce epoxy groups on the slide glass.

(3) Immobilization of Single-stranded Nucleic Acid Probes

Using a DNA synthesizer (manufactured by Applied Biosystem, model 394 DNA synthesizer), nucleic acid probe 2 in which an amino group was introduced was synthesized, and the probe was then purified by high performance liquid chromatography. Next, 5 μl synthesized/purified probes having a concentration of 10 μM and 5 μl potassium hydroxide solution having a concentration of 0 .2 M were mixed to prepare a spotting solution. Furthermore, the prepared spotting solution was spotted at a randomly chosen point on the slide glass using a spotter (manufactured by Hitachi Software, SPBIO 2000), and then the slide glass was left for 6 hours under 37° C. saturated steam to immobilize the nucleic acid probes on the slide glass.

Nucleic Acid Probe 2:

NH₂—(CH₂)₆—O—PO₂—O—5′-GACACAGCAGGTCAAGAGGAGTACA-3′ (SEQ ID NO:1)

(4) Introduction of Functional Groups that can have Negative Charge

The slide glass on which nucleic acid probes were immobilized was immersed in 100 mM DL-α-alanine (Wako Pure Chemical Industries, Ltd.) at 37° C., which was adjusted to pH 9.0 with a CHES buffer solution (N-Cyclohexyl-2-aminoethanesulfonic Acid; 10 mM).

In this example, in contrast to Examples 1-6, functional groups that can react with the functional groups of single-stranded nucleic acid probes, were introduced on a substrate, and then single-stranded nucleic probes were directly immobilized there to without using a crosslinker. Subsequently, in the same manner as in Example 1, a carboxyl group that can have a negative charge by dissociating in a solution was introduced by covalent bond to the surface of a region where no nucleic acid probe had been immobilized. Due to a similar effect as that described in Example 1, the compatibility between a high hybridization signal and a low background signal was achieved.

### Example 8

Example 8 was conducted by the same steps as in Example 7 except that step (4) “introduction of a functional group that can have negative charge” was performed in the same manner as in Example 2.

In this example, after single-stranded nucleic acid probes were directly immobilized on a substrate without using a crosslinker as in Example 7, a hydrogensulfate group that can have a negative charge by dissociating in a solution was introduced on the surface of a region where no single-stranded nucleic acid probe had been immobilized in the same manner as in Example 2. Similar to Example 1, both high hybridization signal and low background signal were achieved.

### Example 9

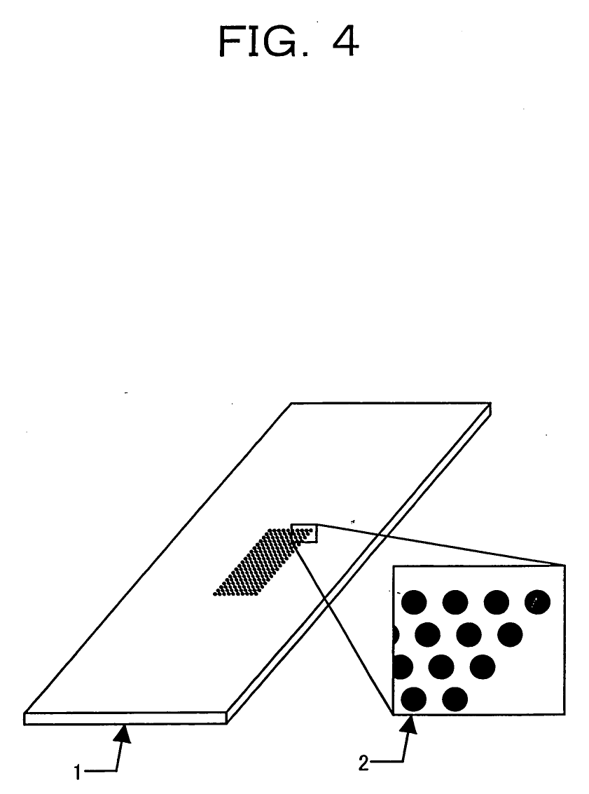

Using-the method comprising the steps (1)-(4) described in Example 4, nucleic acid arrays in which 200 varieties of single-stranded nucleic acid probes were immobilized per slide glass were prepared as shown in FIG. 4. As a nucleic acid probe, a single-stranded nucleic acid probe of 25-base length in which the terminus was modified by a thiol group, the probe being synthesized by the method described in Example 1, was used. Furthermore, as base sequences of the above-mentioned 200 varieties of nucleic acid probes, the inherent consecutive 25-base sequences of respective gene fragments derived from the 200 varieties shown in Tables 1-8 were used.

TABLE 1
Genes used as nucleic acid probes (1)

| GenBank | |
|---|---|
| No. | Gene Name |
| A03911 | Homo sapiens mRNA for glia-derived |
| | neurite-promoting factor (GdNPF) |
| A26792 | Homo sapiens CNTF coding sequence (form b + c) (comp.) |
| AB003791 | Homo sapiens mRNA for keratan sulfate |
| | Gal-6-sulfotransferase |
| AB012192 | Homo sapiens mRNA for chondroitin |
| | 6-sulfotransferase |
| AF000546 | Homo sapiens purinergic receptor P2Y5 mRNA |
| AF000974 | Human zyxin related protein ZRP-1 mRNA |
| AF001954 | Homo sapiens growth inhibitor p33ING1 (ING1) mRNA |
| AF004430 | Homo sapiens hD54 + ins2 isoform (hD54) mRNA |
| AF007111 | Homo sapiens MDM2-like p53-binding protein (MDMX) |
| | mRNA |
| AF009674 | Homo sapiens axin (AXIN) mRNA |
| AF010127 | Homo sapiens Casper mRNA |
| AF010310 | Homo sapiens p53 induced protein mRNA partial cds |
| AF013168 | Homo sapiens hamartin (TSC1) mRNA |
| AF015950 | Homo sapiens telomerase reverse transcriptase |
| | (hTRT) mRNA |
| AF016267 | Homo sapiens TRAIL receptor 3 mRNA |
| AF016268 | Homo sapiens death receptor 5 (DR5) mRNA |
| AF016582 | Homo sapiens checkpoint kinase Chk1 (CHK1) mRNA |
| AF018253 | Homo sapiens receptor activator of nuclear |
| | factor-kappa B (RANK) mRNA |
| AF019770 | Homo sapiens macrophage inhibitory cytokine-1 |
| | (MIC-1) mRNA |
| AF019952 | Homo sapiens tumor suppressing STF cDNA 1 (TSSC1) |
| | mRNA |
| AF022109 | Homo sapiens HsCdc18p (HsCdc18) mRNA |
| AF022224 | Homo sapiens Bcl-2-binding protein (BAG-1) mRNA |
| AF026816 | Homo sapiens putative oncogene protein mRNA partial |
| | cds |
| AF029403 | Homo sapiens oxysterol 7alpha-hydroxylase (CYP7b1) |
| | mRNA |
| AF037195 | Homo sapiens regulator of G protein signaling RGS14 |
| | mRNA |
| AF038009 | Homo sapiens tyrosylprotein sulfotransferase-1 mRNA |
| AF040705 | Homo sapiens putative tumor suppressor protein |
| | unspliced form (Fus-2) mRNA |
| AF040707 | Homo sapiens candidate tumor suppressor gene 21 |
| | protein isoform I mRNA |

TABLE 2
Genes used as nucleic acid probes (2)

| GenBank | |
|---|---|
| No. | Gene Name |
| AF043254 | Homo sapiens heat shock protein 75 (hsp75) mRNA |
| AF049891 | Homo sapiens tyrosylprotein sulfotransferase-2 |
| | mRNA |
| AF053712 | Homo sapiens osteoprotegerin ligand mRNA |
| AF055584 | Homo sapiens SULT1C sulfotransferase (SULT1C) mRNA |
| AF059195 | Homo sapiens basic-leucine zipper transcription |
| | factor MafG (MAFG) mRNA |
| AF061836 | Homo sapiens putative tumor suppressor protein |
| | (RDA32) mRNA |
| AF067512 | Homo sapiens PITSLRE protein kinase alpha SV1 isoform |
| | (CDC2L1) mRNA |
| AF067519 | Homo sapiens PITSLRE protein kinase beta SV1 isoform |
| | (CDC2L2) mRNA |
| AF070594 | Homo sapiens clone 24570 HNK-1 sulfotransferase mRNA |
| AF087017 | Homo sapiens H19 gene complete sequence |
| AF090318 | Homo sapiens sterol 12-alpha hydroxylase CYP8B1 |
| | (Cyp8b1) mRNA |
| AF112219 | Homo sapiens esterase D mRNA |
| AF188698 | Homo sapiens sulfotransferase-like protein mRNA |
| AF237982 | Homo sapiens oxysterol 7alpha-hydroxylase |
| | (CYP39A1) mRNA |
| AI445492 | NCI_CGAP_Gas4 Homo sapiens cDNA clone IMAGE: 2142448 |
| | 3′ mRNA sequence |
| AJ004832 | Homo sapiens mRNA for neuropathy target esterase |
| AL021878 | Human CYP2D7AP |
| AL021878 | Human CYP2D8P |
| D14012 | Human mRNA for hepatocyte growth factor (HGF) |
| | activator precursor |
| D14497 | Human mRNA for proto-oncogene protein |
| D14838 | Human mRNA for FGF-9 |
| D14889 | Human mRNA for small GTP-binding protein S10 |
| D16234 | Human mRNA for phospholipase C-alpha |
| D26512 | Human mRNA for membrane type matrix |
| | metalloproteinase |
| D37965 | Human mRNA for PDGF receptor beta-like tumor |
| | suppressor (PRLTS) |

TABLE 3
Genes used as nucleic acid probes (3)

| GenBank | |
|---|---|
| No. | Gene Name |
| D38122 | Human mRNA for Fas ligand |
| D38305 | Human mRNA for Tob |
| D43968 | Human AML1 mRNA for AML1b protein (alternatively |
| | spliced product) |
| D49742 | Human mRNA for HGF activator like protein |
| D50310 | Human mRNA for cyclin I |
| D86640 | Homo sapiens mRNA for stac, complete cds |
| D88667 | Homo sapiens mRNA for cerebroside sulfotransferase |
| D89479 | Homo sapiens mRNA for ST1B2 |
| D89667 | Homo sapiens mRNA for c-myc binding protein |
| D90224 | Human mRNA for glycoprotein 34 (gp34) |
| J02625 | Human cytochrome P-450j mRNA |
| J02871 | Human lung cytochrome P450 (IV subfamily) BI protein |
| J02906 | Human cytochrome P450IIF1 protein (CYP2F) mRNA |
| J02958 | Human MET proto-oncogene mRNA |
| J03210 | Human collagenase type IV mRNA 3′ end |
| J03241 | Human transforming growth factor-beta 3 |
| | (TGF-beta3) mRNA |
| J03518 | Human epoxide hydrolase microsomal (xenobiotic) |
| | (EPHX1) mRNA |
| J03528 | Human cation-independent mannose 6-phosphate |
| | receptor mRNA |
| J03817 | Human glutathione transferase M1B (GST1) mRNA |
| J03934 | Human, NAD(P)H: menadione oxidoreductase mRNA |
| J04093 | Homo sapiens phenol UDP-glucuronosyltransferase |
| | (UDPGT) mRNA |
| J04127 | Human aromatase system cytochrome P-450 (P450XIX) |
| | mRNA |
| J05070 | Human type IV collagenase mRNA |
| J05459 | Human glutathione transferase M3 (GSTM3) mRNA |
| K01171 | Human HLA-DR alpha-chain mRNA |
| K02276 | Human (Daudi) translocated t (8;14) c-myc oncogene |
| | mRNA |
| K03191 | Human cytochrome P-1-450 (TCDD-inducible) mRNA |
| K03222 | Human (cell line 1027 F57) transforming growth |
| | factor-alpha mRNA |
| L03840 | Human fibroblast growth factor receptor 4 (FGFR4) |
| | mRNA |

TABLE 4
Genes used as nucleic acid probes (4)

| GenBank | |
|---|---|
| No. | Gene Name |
| L04288 | Homo sapiens cyclophilin-related protein mRNA |
| L04751 | Human cytochrome p-450 4A (CYP4A) mRNA |
| L05779 | Human cytosolic epoxide hydrolase mRNA |
| L06895 | Homo sapiens antagonizer of myc transcriptional |
| | activity (Mad) mRNA |
| L07594 | Human transforming growth factor-beta type III |
| | receptor (TGF-beta) mRNA |
| L07765 | Human carboxylesterase mRNA |
| L07868 | Homo sapiens receptor tyrosine kinase (ERBB4) gene |
| L09753 | Homo sapiens CD30 ligand mRNA |
| L11353 | Human moesin-ezrin-radixin-like protein mRNA |
| L12260 | Human recombinant glial growth factor 2 mRNA and |
| | flanking regions |
| L12964 | Human activation dependent T cell mRNA |
| L13286 | Human mitochondrial 125-dihydroxyvitamin D3 |
| | 24-hydroxylase mRNA |
| L13972 | Homo sapiens beta-galactoside |
| | alpha-23-sialyltransferase (SIAT4A) mRNA |
| L15409 | Homo sapiens von Hippel-Lindau disease tumor |
| | suppressor mRNA sequence |
| L17075 | Human TGF-b superfamily receptor type I mRNA |
| L19063 | Human glial-derived neurotrophic factor gene |
| L19067 | Human NF-kappa-B transcription factor p65 subunit |
| | mRNA |
| L20320 | Human protein serine/threonine kinase stk1 mRNA |
| L22005 | Human ubiquitin conjugating enzyme mRNA |
| L22474 | Human Bax beta mRNA |
| L25610 | Homo sapiens cyclin-dependent kinase inhibitor mRNA |
| L25676 | Homo sapiens CDC2-related kinase (PITALRE) mRNA |
| L25851 | Homo sapiens integrin alpha E precursor mRNA |
| L27211 | Human CDK4-inhibitor (p16-INK4) mRNA |
| L29216 | Homo sapiens clk2 mRNA |

TABLE 5
Genes used as nucleic acid probes (5)

| GenBank | |
|---|---|
| No. | Gene Name |
| L29220 | Homo sapiens clk3 mRNA |
| L29222 | Homo sapiens clk1 mRNA |
| L29277 | Homo sapiens DNA-binding protein (APRF) mRNA |
| L32179 | Human arylacetamide deacetylase mRNA |
| L33264 | Homo sapiens CDC2-related protein kinase (PISSLRE) |
| | mRNA |
| L35253 | Human p38 mitogen activated protein (MAP) kinase |
| | mRNA |
| L40027 | Homo sapiens glycogen synthase kinase 3 mRNA |
| L78440 | Homo sapiens STAT4 mRNA |
| M10988 | Human tumor necrosis factor (TNF) mRNA |
| M11730 | Human tyrosine kinase-type receptor (HER2) mRNA |
| M12272 | Homo sapiens alcohol dehydrogenase class I gamma |
| | subunit (ADH3) mRNA |
| M12783 | Human c-sis/platelet-derived growth factor 2 |
| | (SIS/PDGF2) mRNA |
| M12963 | Human class I alcohol dehydrogenase (ADH1) alpha |
| | subunit mRNA |
| M13194 | Human excision repair protein (ERCC1) mRNA clone |
| | pcDE |
| M13228 | Human N-myc oncogene protein mRNA |
| M13755 | Human interferon-induced 17-kDa/15-kDa protein |
| | mRNA |
| M14505 | Human (clone PSK-J3) cyclin-dependent protein |
| | kinase mRNA |
| M14564 | Human cytochrome P450c17 (steroid |
| | 17-alpha-hydroxylase/1720 lyase) mRNA |
| M14695 | Human p53 cellular tumor antigen mRNA |
| M14745 | Human bcl-2 mRNA |
| M14764 | Human nerve growth factor receptor mRNA |
| M15024 | Human c-myb mRNA |
| M15400 | Human retinoblastoma susceptibility mRNA |
| M16038 | Human lyn mRNA encoding a tyrosine kinase |
| M17016 | Human serine protease-like protein mRNA |
| M17252 | Human cytochrome P450c21 mRNA 3′ end |
| M18112 | Human poly(ADP-ribose) polymerase mRNA |

TABLE 6
Genes used as nucleic acid probes (6)

| GenBank | |
|---|---|
| No. | Gene Name |
| M18737 | Human Hanukah factor serine protease (HuHF) mRNA |
| | (cytotoxic T-lymphocyte-associated serine esterase |
| | 3) |
| M19154 | Human transforming growth factor-beta-2 mRNA |
| M19720 | Human L-myc protein gene |
| M19722 | Human fgr proto-oncogene encoded p55-c-fgr protein |
| M20403 | Human cytochrome P450 db1 mRNA |
| M21574 | Human platelet-derived growth factor receptor alpha |
| | (PDGFRA) mRNA |
| M21616 | Human platelet-derived growth factor (PDGF) |
| | receptor mRNA |
| M21758 | Human glutathione S-transferase A2 (GSTA2) mRNA |
| M22995 | Human ras-related protein (Krev-1) mRNA |
| M23619 | Human HMG-I protein isoform mRNA (HMGI gene) clone |
| | 6A |
| M24898 | Human triiodothyronine recptor (THRA1 ear1) mRNA |
| M25753 | Human cyclin B mRNA 3′ end |
| M26880 | Human ubiquitin mRNA |
| M27968 | Human basic fibroblast growth factor (FGF) mRNA |
| M28209 | Homo sapiens GTP-binding protein (RAB1) mRNA |
| M28211 | Homo sapiens GTP-binding protein (RAB4) mRNA |
| M28215 | Homo sapiens GTP-binding protein (RAB5) mRNA |
| M29366 | Human epidermal growth factor receptor (ERBB3) mRNA |
| M29870 | Human ras-related C3 botulinum toxin substrate (rac) |
| | mRNA variant 1 |
| M30496 | Human ubiquitin carboxyl-terminal hydrolase (PGP |
| | 9.5, UCH-L3) isozyme L3 mRNA |
| M30817 | Human interferon-induced cellular resistance |
| | mediator protein (MxA) mRNA |
| M30818 | Human interferon-induced cellular resistance |
| | mediator protein (MxB) mRNA |
| M31165 | Human tumor necrosis factor-inducible (TSG-6) mRNA |
| | fragment adhesion receptor CD44 putative CDS |
| M31899 | Human DNA repair helicase (ERCC3) mRNA |

TABLE 7
Genes used as nucleic acid probes (7)

| GenBank | |
|---|---|
| No. | Gene Name |
| M32977 | Human heparin-binding vascular endothelial growth |
| | factor (VEGF) mRNA |
| M33318 | Human cytochrome P450IIA3 (CYP2A3) mRNA |
| M34065 | Human cdc25Hs mRNA |
| M34309 | Human epidermal growth factor-receptor (HER3) mRNA |
| M34641 | Human fibroblast growth factor (FGF) receptor-1 mRNA |
| M35296 | Human tyrosine kinase arg gene mRNA |
| M35410 | Human insulin-like growth factor binding protein |
| | 2 (IGFBP2) mRNA |
| M35416 | Human GTP-binding protein (RALB) mRNA |
| M35543 | Human GTP-binding protein (G25K) mRNA |
| M36542 | Human lymphoid-specific transcription factor mRNA |
| M36981 | Human putative NDP kinase (nm23-H2S) mRNA |
| M37825 | Human fibroblast growth factor-5 (FGF-5) mRNA |
| M54915 | Human h-pim-1 protein (h-pim-1) mRNA |
| M54968 | Human K-ras oncogene protein mRNA |
| M55618 | Homo sapiens hexabrachion (HXB) mRNA |
| M57230 | Human membrane glycoprotein gp130 mRNA |
| M57732 | Human hepatic nuclear factor 1 (TCF1) mRNA |
| M58051 | Human fibroblast growth factor receptor (FGFR3) mRNA |
| M58525 | Homo sapiens catechol-O-methyltransferase (COMT) |
| | mRNA |
| M59040 | Human cell adhesion molecule (CD44) mRNA |
| M59465 | Human tumor necrosis factor alpha inducible protein |
| | A20 mRNA |
| M59964 | Human stem cell factor mRNA |
| M60278 | Human heparin-binding EGF-like growth factor mRNA |
| M60614 | Human Wilms' tumor (WIT-1) associated protein mRNA |
| M60618 | Human nuclear autoantigen (SP-100) mRNA |
| M60718 | Human hepatocyte growth factor mRNA |
| M60828 | Human keratinocyte growth factor mRNA |
| M60854 | Human ribosomal protein S16 mRNA |
| M60915 | Human neurofibromatosis protein type I (NF1) mRNA |

TABLE 8
Genes used as nucleic acid probes (8)

| GenBank | |
|---|---|
| No. | Gene Name |
| M60974 | Human growth arrest and DNA-damage-inducible |
| | protein (gadd45) mRNA |
| M61176 | Homo sapiens brain-derived neurotrophic factor |
| | precursor (BDNF) mRNA |
| M61853 | Human cytochrome P4502C18 (CYP2C18) mRNA clone 6b |
| M61854 | Human cytochrome P4502C19 (CYP2C19) mRNA clone 11a |
| M61857 | Human cytochrome P4502C9 (CYP2C9) mRNA clone 65 |
| M62401 | Human sterol 27-hydroxylase (CYP27) mRNA |
| M62829 | Human transcription factor ETR103 mRNA |
| M63167 | Human rac protein kinase alpha mRNA |
| M64240 | Human helix-loop-helix zipper protein (max) mRNA |
| M64349 | Human cyclin D (cyclin D1) mRNA |
| M68520 | Human cdc2-related protein kinase mRNA |
| M73791 | Human novel gene mRNA |
| M73812 | Human cyclin E mRNA sequence |

Next, a hybridization solution was prepared by the following method.

Approximately 2×10⁶ pancreatic cancer cells (American Type Culture Collection, CFPAC1) and 10 ml of medium were added in a dish, and the cells were cultured for 1 week at 37° C. while exchanging the medium once every two days. As a medium, a 9:1 mixture of D-MEM (LIFETEC ORIENTAL) and Fetal Bovine Serum, Qualified (LIFETEC ORIENTAL) was used. After culturing, the medium was removed from the dish, and GTC solution (guanidine thiocyanate; 4M, Tris (hydroxymethyl) aminomethane; 0.1 M, 2-mercaptoethanol; 1%, pH 7.5) was added therein to dissolve the cultured cells. Next, sodium N-lauroyl sarcosinate was added therein to a final concentration of 0.5%, followed by centrifugation for 10 min at 5,000 r.p.m., after which its supernatant was taken out. 5.7 M cesium chloride solution was added to the obtained supernatant such that the ratio of the supernatant to the cesium chloride solution was 7:3. The mixture was subjected to centrifugation for 12 hours at 35,000 r.p.m. with further addition of an appropriate amount of light liquid paraffin. After centrifugation, RNA pellet precipitated in a lower layer was taken out. After the obtained RNA pellet was dissolved in an appropriate amount of TES solution (Tris (hydroxymethyl) aminomethane; 10 mM, ethylenediaminetetraacetic acid; 5 mM, sodium dodecyl sulfate; 1%, pH 7.4), ethanol precipitation was performed to concentrate and purify the RNA pellet. Next, the purified RNA pellet was dissolved in DEPC solution (diethyl dioxide; 0.1%), and then mRNAs were collected from the RNA pellet using an m-RNA purification kit (Invitrogen, Micro-Fast Track 2.0 Kit). After the obtained mRNAs were diluted to 1 μg/μ1, 1 μl of 0.5 μg/μl Oligo dT primer (LIFETEC ORIENTAL) and 5 μl DEPC solution were added to 1 μl of the diluted solution, and the solution was kept warm for 5 min at 70° C. Subsequently, to 5 μl of the obtained solution, 5 μl of SuperScript II buffer (LIFETEC ORIENTAL, Super Script II Reverse Transcriptase), 2 μl of dNTP mixture (2 mM dUTP, 5 mM dATP, 5 mM dGTP, 5 mM dCTP), 2 μl of 100 mM DTT (dithiothreitol), 2.5 μl of 40U Rnasin (TOYOBO, Rnase inhibitor), 2 μl of 1 mM FluoroLink dUTP (Amersham Pharmacia, FluoroLink Cy5-dUTP) and 1 μl of SS II (LIFETEC ORIENTAL, Super Script II Reverse Transcriptase) were mixed, and then the solution was kept warm for 30 min at 42° C. Subsequently, 1 μl of SS II (LIFETEC ORIENTAL, Super Script II Reverse Transcriptase) was further added therein, and the solution was kept warm again for 30 min at 42° C. To the warmed solution, 20 μl of DEPC solution, 5 μl of 0.5 M ethylene diamine tetraacetic acid and 10 μl of 1N sodium hydroxide solution were added and the solution was kept warm for 60 min at 65° C. Then, 25 μl of 1 M Tris (hydroxymethyl) aminomethane buffer solution (pH 7.5) was added to neutralize the solution. Subsequently, the neutralized sample solution was put in Microcon-30 (Amicon) and subjected to centrifugation for 4 min at 8,000 r.p.m., after which the solution was concentrated to 10-20 μl and unreacted dNTP was removed. The obtained solution, 20×Denhardt's solution (SIGMA), 20×SSC and sodium dodecyl sulfate were mixed appropriately to prepare 24.5 μl of hybridization solution in which the final concentration of each component would be 100 pg/μl nucleic acid, 2×Denhardt's solution, 4×SSC, and 0.2% sodium dodecyl sulfate, respectively.

Next, using the nucleic acid arrays and the hybridization solution obtained by the above method, a hybridization reaction was performed as follows.

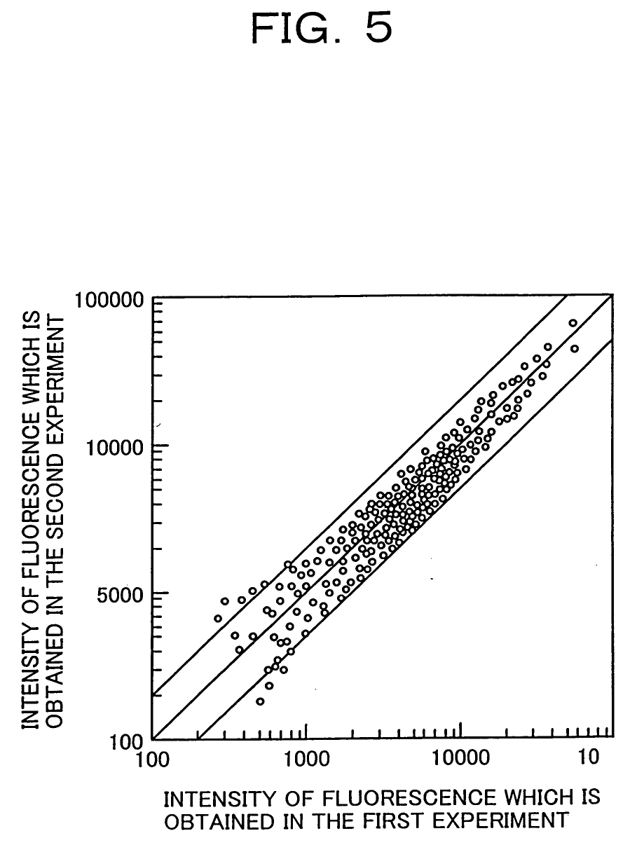

After thermal denaturation of the hybridization solution for one minute at 95° C., the hybridization solution was dropped on a slide glass, and then a cover glass was put thereon. Subsequently, the slide glass was left in a thermostat for 12 hours at 40° C. to carry out a hybridization reaction. After the hybridization reaction, the slide glass was immersed in the mixture of a 10-fold diluted solution of 20×SSC and a 300-fold diluted solution of 10% sodium dodecyl sulfate solution, and the cover glass was then removed. Subsequently, the slide glass was washed with a 100-fold diluted solution of 20×SSC. Next, after water on the slide glass was removed using a centrifugal separator for microtiter plates, the intensity of fluorescence of 200 spots (hybridization signal) and the intensity of fluorescence of a region where no nucleic acid probe was immobilized (background signal) were measured using a scanner for a microarray (GSI Lumonics, Scan Array 5000). For each spot, the background signal was subtracted from the obtained hybridization signal to determine the expression level of the 200 spots. The above hybridization reaction was performed twice in total. Then, for each-spot, the expression level obtained in the first reaction was located on a horizontal axis and that obtained in the second reaction was located on a vertical axis, thereby obtaining the Scatter plot shown in FIG. 5.

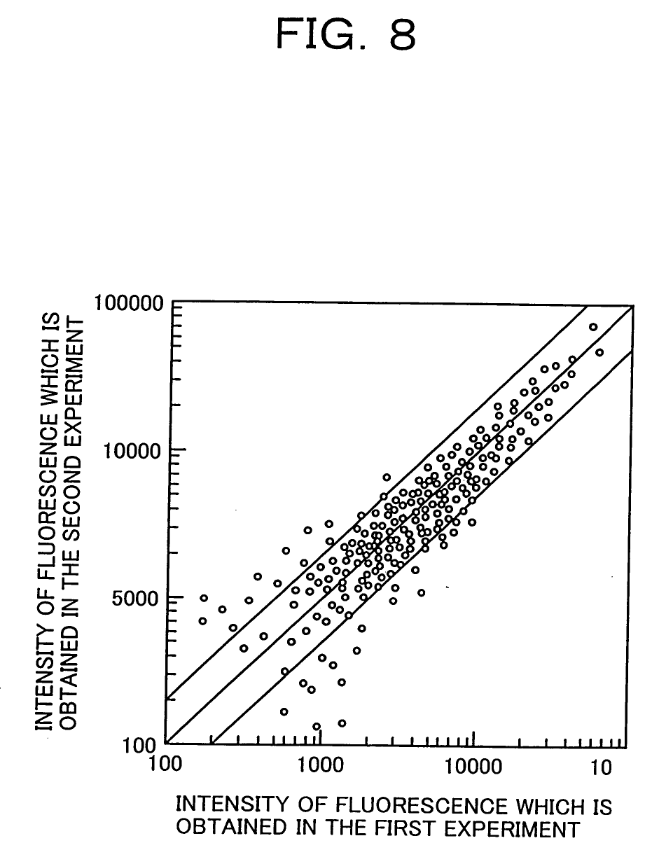

In this example, arrays in which single-stranded nucleic acid probes were immobilized by covalent bond, and a functional group that can have a negative charge by dissociating in a solution was introduced to the surface of a region where no nucleic acid probe was immobilized were prepared. Using the arrays, the gene expression in a pancreatic cancer cell was profiled and the reproducibility of analyzed data was confirmed. Since the arrays of this example achieve the compatibility of a high hybridization signal and a low background signal, the sensitivity for detecting a nucleic acid has been enhanced. And as clearly seen from a comparison between FIG. 5 showing results of this example and FIG. 8 showing results of comparative example 4, this effect enabled minimization of the dispersion of reproducibility at a region where the expression level is low with an intensity of fluorescence of not more than 1,000.

### Example 10

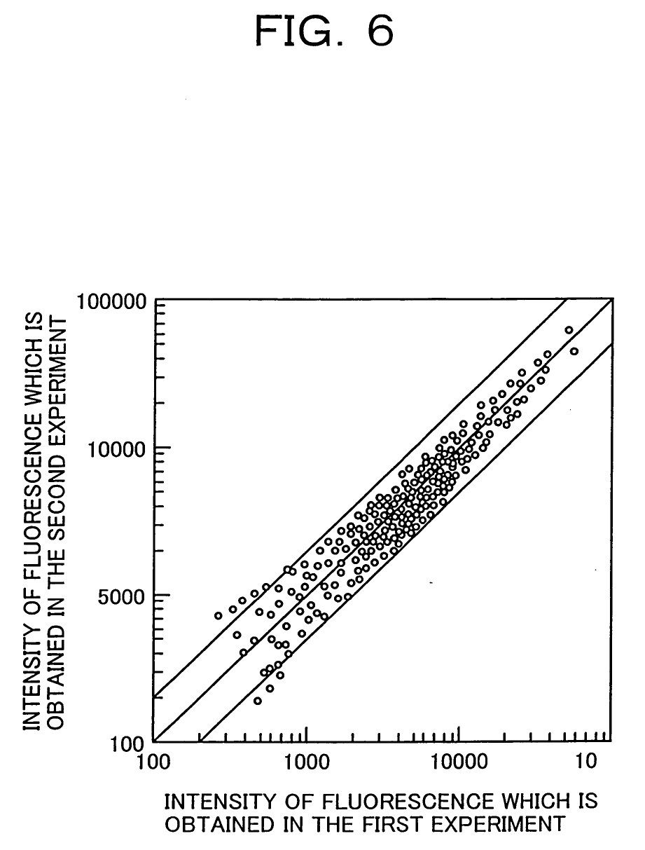

Using the methods comprising the steps (1)-(4) described in Example 5, nucleic acid arrays on which 200 varieties of single-stranded nucleic acid probes were immobilized per slide glass were prepared as shown in FIG. 4. Nucleic acid probes and a hybridization solution as described in Example 9 were used, and a hybridization reaction was also performed in the same manner as in Example 9. The results obtained are shown in FIG. 6.

In this example, arrays were prepared by the method described in Example 5, and expression profile and reproducibility confirmation were performed according to the method described in Example 9. In this example, the dispersion of reproducibility could be minimized due to the same effect as in Example 9.

### Example 11

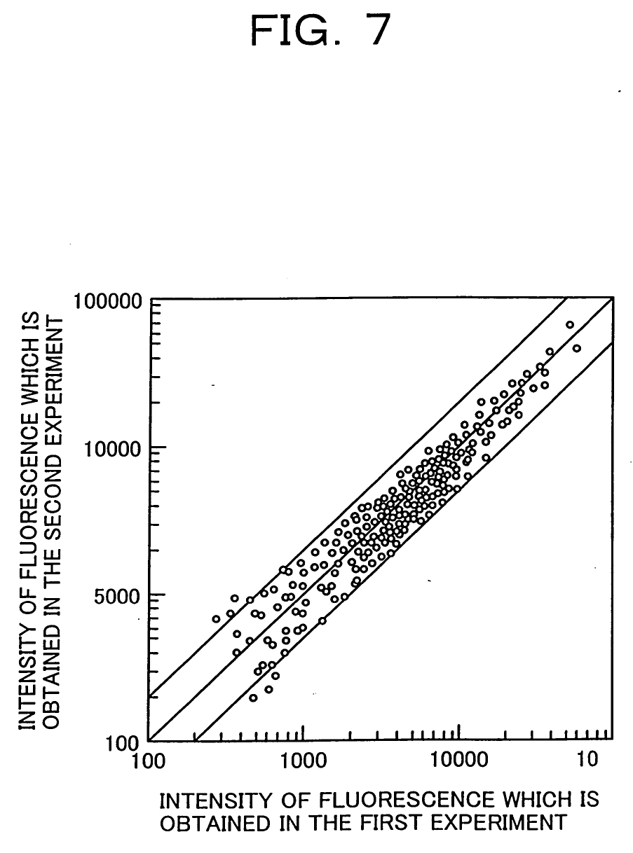

Using the methods comprising the steps (1)-(4) described in Example 6, nucleic acid arrays on which 200 varieties of single-stranded nucleic acid probes were immobilized per slide glass were prepared as shown in FIG. 4. Nucleic acid probes and hybridization solution described in Example 9 were used, and a hybridization reaction was also performed in the same manner as in Example 9. The results obtained are shown in FIG. 7.

In this example, arrays were prepared by the method described in Example 6, and expression profile and reproducibility confirmation were performed according to the method described in Example 9. In this example, the dispersion of reproducibility could be minimized due to the same effect as in Example 9.

### Comparative Example 1

(1) Washing of a Substrate

A commercially available slide glass (Gold Seal Brand; 3010) was immersed in an alkaline solution (sodium hydroxide; 50 g, distilled water; 150 ml, 95% ethanol; 200 ml) for 2 hours at room temperature. Then, the glass was moved into distilled water and rinsed three times, thereby completely removing the alkaline solution.

(2) Introduction of Functional Groups for Immobilizing Double-stranded cDNA Probes

The washed slide glass was immersed in 10% poly-L-lysine (Sigma; P8920) solution for 1 hour, and then the slide glass was taken out and subjected to centrifugation for one minute at 500 r.p.m. using a centrifugal separator for microtiter plates to remove the poly-L-lysine solution. Subsequently, the slide glass was put in a suction thermostat and dried for 5 min at 40° C. to introduce amino groups thereon.

(3) Immobilization of Double-stranded cDNA Probes

Using a plasmid DNA as a template, double-stranded cDNA probes having the sequence shown below were prepared by PCR method. Next, cDNA probes thus—prepared and dimethyl sulfoxide were mixed to prepare a spotting solution (cDNA probe; 0.1 μg/μl, dimethyl sulfoxide; 50%), and the obtained spotting solution was spotted at a randomly chosen point on the slide glass using a spotter (Hitachi Software, SPBIO 2000).

Sequence of Double-stranded cDNA Probe:

| | |
|---|---|
| GGTCGGTTTCAGGAATTTCAAAAGAAATCTGACGTCAATGCAATTATCCATTATTTAAA | (SEQ ID NO: 2) |
| AGCTATAAAAATAGAACAGGCATCATTAACAAGGGATAAAAGTATCAATTCTTTGAAGA | |
| AATTGGTTTTAAGGAAACTTCGGAGAAAGGCATTAGATCTGGAAAGCTTGAGCCTCCTT | |
| GGGTTCGTCTATAAATTGGAAGGAAATATGAATGAAGCCCTGGAGTTACTATGAGCGGG | |
| CCCTGAGACTGGCTGCTGACTTTGAGAACTCTGTGAGACAAGGTCCTTAGGCACCCAGA | |
| TATCAGCC | |

(4) Blocking Process

The slide glass on which cDNA probes were spotted was retained for one minute on a tray containing 60° C. distilled water, and then put on a 95° C. hot plate until the steam cloud disappeared. Subsequently, the slide glass was irradiated with 60 mJ by a UV crosslinker, and then immersed for 15 min in a blocking solution (succinic anhydride; 5 g, N-methyl-pyrrolidinone; 315 ml, 0.2 M sodium tetraborate; 35 ml). After being removed from the blocking solution, the slide glass was immersed in 95° C. distilled water for 2 min and then in 95% ethanol for one minute. Subsequently, the slide glass was subjected to centrifugation for one minute at 500 r.p.m. using a centrifugal separator for microtiter plates to remove ethanol on the slide glass.

(5) Hybridization Reaction

Using a reverse transcription reaction, nucleic acid in which Cy3 having a complementary base sequence to that of the above cDNA probe was taken in, was prepared. The obtained nucleic acid, 20×SSC and 10% sodium dodecyl sulfate were mixed appropriately to prepare a hybridization solution (nucleic acid; 100 pg/μl, 3.4×SSC, sodium dodecyl sulfate; 0.3%). Subsequently, after the thus-prepared hybridization solution was dropped on the slide glass and a cover glass was put thereon, it was left in a thermostat for 12 hours at 62° C. to perform a hybridization reaction. After the hybridization reaction, the slide glass was immersed in the mixture of 10-fold diluted solution of 20×SSC and 300-fold diluted solution of 10% sodium dodecyl sulfate and the cover glass was removed, and then the slide glass was washed with 100-fold diluted solution of 20×SSC. Finally, after water on the slide glass was removed using a centrifugal separator for microtiter plates, the intensity of fluorescence of a region where cDNA probes were immobilized (hybridization signal) and the intensity of fluorescence of a region where no cDNA probe was immobilized (background signal) were measured using a scanner for a micro array (GSI Lumonics, Scan Array 5000). The results are shown in FIG. 2 and FIG. 3.

In this comparative example, arrays in which double-stranded cDNA probes were electrostatically bound on a substrate were prepared, and comparison was made to those described in examples. In the comparative example, since nucleic acid probes are stripped during the blocking process or hybridization, the hybridization signal decreased. Further, because of the inadequacy of the blocking process, the background signal increased.

### Comparative Example 2

Comparative example 2 was conducted by the same steps as in Example 4 except that step (4) “introduction of a functional group that can have negative charge” was altered to (4′) “blocking process”, as follows.

(4′) Blocking Process

A blocking solution of 10 mg/ml of Bovine Serum Albumin (SIGMA, ALUBUMIN BOVINE) with a SSC concentration of 3.5×SSC was prepared. The slide glass on which nucleic acid probes were immobilized was immersed for 6 hours in the 40° C. blocking solution.

In this comparative example, after single-stranded nucleic acid probes were immobilized by covalent bond, Bovine Serum Albumin was introduced into a region where no nucleic acid probe was immobilized, thereby performing a blocking process to prevent adsorption of nucleic acid. Although the background signal slightly weakened due to the introduction of Bovine Serum Albumin, the hybridization signal decreased since the molecular weight of Bovine Serum Albumin is large and steric hindrance is created when nucleic acids approach a nucleic acid probe.

### Comparative Example 3

Comparative example 3 was conducted by the same steps as in Example 4 except that step (4) “introduction of functional groups that can have negative charge” was altered to (4′) “blocking process,” as follows.

(4′) Blocking Process

The slide glass on which nucleic acid probes were immobilized was immersed for two hours in a 100 mM 2-mercaptoethanol (Wako Pure Chemical Industries, Ltd.) solution in which the pH was adjusted to 6.5 with HEPES buffer solution.

In this comparative example, after single-stranded nucleic acid probes were immobilized by covalent bond, an alcoholic hydroxyl group was introduced into a region where no nucleic acid probe was immobilized using 2-mercaptoethanol, thereby performing a blocking process to prevent adsorption of nucleic acids. Stripping could be prevented due to the immobilization of single-stranded nucleic acid probes by covalent bond and a high hybridization signal could be obtained. However, since the introduced alcoholic hydroxyl group was almost neutral in an aqueous solution, blocking efficacy was insufficient and the background signal increased.

### Comparative Example 4

Using the method comprising the steps (1)-(4) described in Comparative example 1, nucleic acid arrays on which 200 varieties of nucleic acid probes were immobilized per slide glass were prepared as shown in FIG. 4. As nucleic acid probes, double-stranded cDNA probes having 200-through 400-base length were prepared using the PCR method as described in Comparative example 1. Furthermore, as the respective base sequences possessed by the 200 varieties of cDNA probes, the inherent consecutive 200- through 400-base sequences of respective gene fragments derived from the 200 varieties shown in Tables 1-8 were used. Next, a hybridization reaction was performed using the hybridization solution described in Example 9. For each spot, the background signal was subtracted from the obtained hybridization signal to determine the expression level of the 200 spots. The above hybridization reaction was performed twice in total. Then, for each spot, the expression level obtained in the first reaction was located on a horizontal axis and that obtained in the second reaction was located on a vertical axis, thereby obtaining the Scatter plot shown in FIG. 8.

In this comparative example, arrays in which double-stranded cDNA probes were electrostatically bound on a substrate in the manner described in Comparative example 1 were prepared, and using the arrays, the gene expression in a pancreatic cancer cell was analyzed and the reproducibility of the analyzed-data was confirmed. Since the arrays of this comparative example have low detection sensitivity for nucleic acids, in the detection of nucleic acids using this, the reproducibility varied widely at a region in which the expression level was low, having an intensity of fluorescence of not more than 1,000.

The present invention further provides additional embodiments as follows:

(1) A method for detecting nucleic acids which comprises detecting a target nucleic acid hybridization using nucleic acid arrays, in which various kinds of single-stranded nucleic acid probes are immobilized by covalent bond at different positions on a substrate, and functional groups which can have negative charge by dissociating in an aqueous solution are present on the surface of regions of the substrate on which no nucleic acid probe is immobilized.

(2) The method for detecting nucleic acids of (1) above, wherein said functional groups which can have negative charge are introduced by the steps comprising:

immobilizing single-stranded nucleic acid probes on a substrate; and immobilizing by covalent bond a compound with the functional groups which can have negative charge onto regions on which no single-stranded nucleic acid probe is immobilized.

(3) The method for detecting nucleic acids of (1) above, wherein said functional groups which can have negative charge are introduced by the steps comprising:

immobilizing single-stranded nucleic acid probes on a substrate;

immobilizing by hydrophobic bond a compound with the functional groups which can have negative charge onto regions on which no single-stranded nucleic acid probe is immobilized.

(4) A method for detecting nucleic acids which comprises detecting a target nucleic acid by hybridization using nucleic acid arrays, in which various kinds of single-stranded nucleic acid probes are immobilized by covalent bond at different positions on a substrate, and functional groups which can have negative charge by hydrolysis are present on the surface of regions of the substrate on which no nucleic acid probe is immobilized.

### ADVANTAGE OF THE INVENTION

As described above, in the present invention, single-stranded nucleic acid probes immobilized on a substrate by covalent bond and nucleic acids are hybridized, thereby preventing stripping of nucleic acid probes, and at the same time, enhancing the efficiency of hybridization to increase the detection volume of nucleic acids. Furthermore, functional groups that can dissociate in a solution and have a negative charge or functional groups that have a negative charge by hydrolysis are introduced into the surface of a region where no nucleic acid probe is immobilized, enabling inhibition of adsorption of nucleic acids to reduce noises. Due to the above two effects, the detection sensitivity for nucleic acids can be enhanced. Moreover, in the detection of nucleic acids the reproducibility of analysis data can be improved and highly reliable analysis data can be obtained with the enhanced detection sensitivity.

What is claimed is:

1. A method for manufacturing a nucleic acid array for detecting nucleic acids by hybridization comprising:
providing a substrate in which maleimide groups are formed on a surface;
immobilizing single-stranded nucleic acid probes for hybridizing to the nucleic acids on a first region of the surface covalently;
after said immobilizing single-stranded nucleic acid probes, hydrolyzing maleimide groups formed on the second region where said single-stranded nucleic acid probes are not immobilized.

2. A method for manufacturing a nucleic acid array according to claim 1, wherein said single-stranded nucleic acid probes are bonded via thioether linkage to said maleimide groups.

3. A method for manufacturing a nucleic acid array according to claim 1, wherein said maleimide groups is hydrolyzed in an alkaline solution.


SEQUENCE LISTING (2 sequences)

SEQ ID NO: 1 - DNA, 25 bp, Artificial Sequence
  Description of Artificial Sequence Nucleic Acid Probe
gacacagcaggtcaagaggagtaca

SEQ ID NO: 2 - DNA, 303 bp, Artificial Sequence
  Description of Artificial Sequence Double Strand cDNA Probe
ggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctataaattggaaggaaatatgaatgaagccctggagttactatgagcgggccctgagactggctgctgactttgagaactctgtgagacaaggtccttaggcacccagatatcagcc